(19) United States
(12) Patent Application Publication
(10) Pub. No.: US 2020/0121520 A1
(43) Pub. Date: Apr. 23, 2020

(54) Infans Compost

(71) Applicant: Crystal Sylver, Valley Springs, CA (US)

(72) Inventor: Crystal Sylver, Valley Springs, CA (US)

(73) Assignee: Crystal Sylver, Valley Springs, CA (US)

(21) Appl. No.: 16/166,088

(22) Filed: Oct. 20, 2018

(51) Int. Cl. A61F 13/505; A61F 13/84; A61F 13/496; A61F 13/15; A61L 15/46; A61L 15/18; A61L 15/28

(52) U.S. Cl. CPC A61F 13/505; A61F 13/84; A61F 13/4963; A61F 13/15252; A61F 2013/49092; A61L 15/46; A61L 15/18; A61L 15/28; A61F 13/8405

(57) ABSTRACT

The invention is a biodegradable, compostable thin cotton baby liner used with the cloth diaper system.

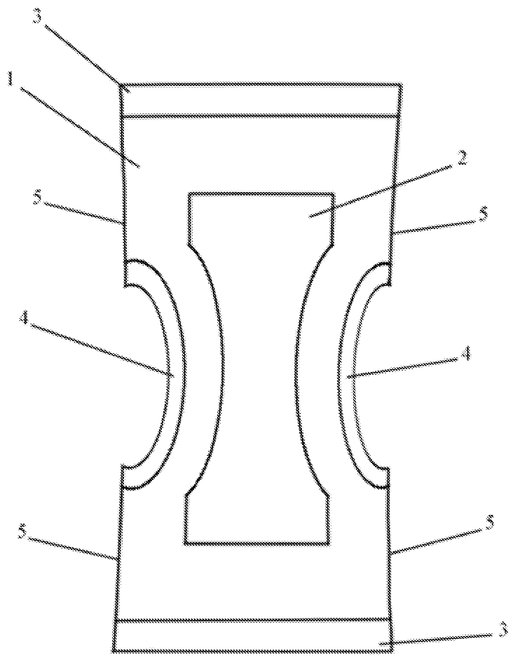

### CROSS-REFERENCE TO RELATED APPLICATIONS

Infans Compost

Provisional Patent Filed Oct. 23, 2017

Application No. 67/707,115

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF INVENTION

The Infans Compost is a manufactured article. It is a compostable baby liner made for the cloth diaper system to be used in place of the disposable diaper system.

The disposable baby diaper system has created billions of tons of nonbiodegradable waste in landfills around the world. Disposable diapers take between 200-500 years to decompose. They are made from plastic and crystal polymers, which are not healthy for babies or the environment.

### BRIEF SUMMARY OF INVENTION

The Infans Compost is a cotton/bamboo biodegradable and compostable baby liner to be used with the cloth diaper system. The advantages being its cleanliness and ease of use compared with the cloth inserts, which must be cleaned off and washed by hand or machine. The compost liner with thick cotton soaker is thrown out and replaced with a new one. This will save parents time and resources, and create a cleaner diaper system.

The Infans Compost used with the cloth diaper system has an advantage over the plastic disposable diaper industry as well. It will save consumers money by using the cloth diaper system, it will save water and resources, and it will reduce landfill waste worldwide. It is also a cleaner and healthier mode of baby diapering, eliminating plastics, crystal polymers, and nonbiodegradable waste. It is healthier for baby's skin to use natural diapers and liners rather than plastics. Plastic disposable diapers can cause heat rash, irritation, and allergies.

It offers advantages to the cloth diaper system as well as the disposable diaper system.

### BRIEF DESCRIPTION OF THE SEVERAL VIEWS OF THE DRAWING

1/1 Infans Compost open top view

### 1. Baby Liner

### 2. Cotton Soaker

### 3. Waistband

### 4. Leg Bands

### 5. Side Seams

1.) 100% cotton compost Baby Diaper Liner made of ruched fibers, 2.) thick 100% Cotton Soaker to absorb baby waste. 3.) Non-elastic Waistband made of ruched cotton fibers, and 4.) Leg Bands made of non-elastic ruched cotton fibers. 5.) Two side seams on either side of the hips from the top of the waistband to the bottom of the leg bands.

### DETAILED DESCRIPTION OF THE INVENTION

The Infans Compost is a thin liner made for the cloth diaper system. It is used as an inner liner to absorb waste and prevent leaks, similar to pull-on diapers or regular cotton underwear, but made of very thin cotton material with a soaker in the middle.

Made of 100% cotton fabric, soft like a tissue and baby wipe combined, it naturally stretches and molds to a baby's body through ruched fibers, creating a stretch material, allowing the skin to breath, and eliminating heat rash and diaper rash. The non-elastic waistband and leg bands are made of ruched cotton fibers that create a natural stretch material and leak guard. The waistband fits at the waist preventing leaks. The soaker is 100% cotton and absorbs liquids, whisks moisture away from baby's skin, and reduces odor with an infusion of talcum powder in the cotton fibers. A thin layer of perforated cotton material covers the soaker to prevent direct contact with skin. The liner comes with the side seams already intact with compostable glue, allowing the liner to be pulled up like underwear. After use the side seams easily pull apart, and the liner opens up into one piece, which allows the liner to be removed quickly and easily. The liner is then rolled into a ball and thrown into the compost. The Infans Compost is covered by a cloth diaper for added leak guard protection, which can be washed, reused and recycled. It can be sized for preemies, newborns, infants, and toddlers.

Currently, the cloth diaper system uses a thick cloth insert for absorbing waste, or none at all. It is messy to clean and can be more time-consuming than disposable diapers. The Infans Compost will streamline the diapering process, making it cleaner and faster, while also utilizing the many health benefits and environmental benefits of the cloth diaper system, guaranteeing more interest from consumers in the cloth diaper method.

The disposable diaper method is not healthy or good for the environment. It creates non-biodegradable waste in landfills around the world, they are made of irritating plastics using crystal polymers to soak up waste, and they do not prevent leaks or blowouts. They are dirty and messy. The convenience of disposable diapers is the ease of use and the ability to throw them out immediately. This convenience has resulted in unsanitary and unhealthy environmental conditions around the world.

The Infans Compost is made similar to an adult diaper in a manufacturing facility that produces perhaps baby diapers, adult diapers, women's underwear liners, women's cotton menstrual pads, cloth diapers, or other baby products. The cotton liner material is infused with a thicker cotton material in the center, this cotton is infused with a talcum powder, and then a thin perforated cotton material is fused to the perimeter area of the cotton soaker. The side seams are pressed together with a compost glue, with the ability to pull apart.

The Infans Compost is pulled onto the baby, which fits the body like form-fitting light cotton underwear. A cotton diaper is then secured over the liner. When the baby needs to be changed, the cotton diaper is removed and thrown in the hamper or reused if there are no leaks. The liner is pulled apart on each side by the two side seams. It opens up and allows the baby to be cleaned and the liner to be rolled up into a small ball and thrown in the designated compost bin or trash. A new liner is then put on the baby and a new or clean cloth diaper is put on over the liner.

This invention can also be used as a replacement for the adult diaper industry, used as a liner for cotton underwear, rather than a bulky plastic adult diaper. Adults would appreciate a liner rather than a diaper. And it can easily be removed and thrown out by pulling the side seams apart. The liner can also be used for older kids who wet the bed or are not fully potting trained and need some extra protection under their regular underwear.

1. The invention claimed is Infans Compost comprised of:
a thin cotton material made of ruched cotton fibers that fits like underwear, a waistband made of ruched cotton fibers that fits at the waist, two leg bands made of ruched cotton fibers, a thick cotton soaker infused with talcum powder and covered by a thin perforated cotton material;
two side seams on either side of the hips from the top of the waistband to the bottom of the leg bands that are pressed together with a light compostable, non-toxic glue;
made for preemies, newborns, infants, and toddlers.

2. The Infans Compost as recited in claim 1, may use bamboo material or other biodegradable, compostable materials in place of cotton or in addition to cotton.

3. The Infans Compost as recited in claim 1, may use a compostable alternative to talcum powder for whisking liquids and odors away from the body, or none at all.

4. The Infans Compost as recited in claim 1, may use various shapes for the cotton soaker best suited to the size of the liner and individuals.

5. The Infans Compost as recited in claim 1, may use any variety of biodegradable tacky substances in order to hold the two side seams together, or other methods that don't require glue, but fuse the seams together.

6. The Infans Compost as recited in claim 1, may use a variety of waistband and leg band widths, depending on the size of liner and individuals.

7. The Infans Compost as recited in claim 1, may have designs on them using non-toxic or plant-based inks, or none at all.

8. The Infans Compost as recited in claim 1, may be made as a liner for adults.

9. The Infans Compost as recited in claim 1, may be made as a liner for older children for bed-wetting, potty training, or extra protection.
